(19) United States
(12) Patent Application Publication
(10) Pub. No.: US 2025/0040538 A1
(43) Pub. Date: Feb. 6, 2025

(54) NOVEL COMPOSITIONS COMPRISING TRANS RETINOL

(71) Applicant: DSM IP ASSETS B.V., Heerlen (NL)

(72) Inventors: Christine MENDROK-EDINGER, Kaiseraugst (CH); Eileen JACKSON, Kaiseraugst (CH); Angela WILDERMANN, Kaiseraugst (CH); Peter Louis HOUSTON, Kaiseraugst (CH)

(21) Appl. No.: 18/715,377

(22) PCT Filed: Dec. 5, 2022
(86) PCT No.: PCT/EP2022/084469

§ 371 Date: May 31, 2024

Related U.S. Application Data
(60) Provisional application No. 63/285,604, filed on Dec. 3, 2021.

(30) Foreign Application Priority Data
Dec. 1, 2021 (EP) 21216502.1
May 5, 2022 (EP) 22171737.4

(51) Int. Cl. A01N 31/04; A01P 1/00; A61K 8/67; A61K 31/07; A61P 31/00; A61Q 17/00

(52) U.S. Cl. CPC A01N 31/04; A01P 1/00; A61K 8/671; A61K 31/07; A61P 31/00; A61Q 17/005

(57) ABSTRACT

The present invention relates to novel compositions comprising mixtures of cis and trans isomers of retinol, particularly with a cis/trans ratio of less than 1:100, as well as the use of said compositions for preservation.

The present invention relates to novel compositions comprising mixtures of cis and trans isomers of retinol, particularly with a cis/trans ratio of less than 1:100, as well as the use of said compositions for preservation.

To protect food, cosmetic or household products, plastics, paper and/or paints against mold, most products currently on the market contain preservatives. While these preservatives protect against fungi, studies have linked daily exposure to many of these substances to an increased risk of skin irritation, cancer and/or endocrine problems. Thus, many manufactures are searching for alternative antimicrobial actives which allow reducing the amount of preservatives and don't appear to pose any health risks.

Surprisingly, it has now been found that certain retinol mixtures comprising cis and trans isomers of retinol, particularly with a ratio of cis- to trans-retinol of about less than 1:100, exhibit excellent antimicrobial activity, particularly activity against fungal cells, more particularly activity against Aspergillus, even more particularly against Aspergillus brasiliensis (A. brasiliensis).

Thus, the present invention relates to a retinol mixture comprising cis and trans isomers of retinol, with a ratio of cis-retinol (wt %) to trans-retinol (wt %) of less than 1:100 (i.e. a ratio of less than 0.01), particularly mixtures of retinol isomers with a cis/trans ratio of about 0.003.

Particularly, the present invention is directed to a pharmaceutical, food, feed or cosmetic composition comprising said mixture with a ratio of cis- to trans-retinol of less than 1:100, including pharmaceutical, food, feed, household or cosmetic compositions that are different from commercially available pure all-trans retinol compositions, such as e.g., retinol marketed from BASF, DSM, ZMC, Adisseo (e.g. Microvit®A), etc.

The term “retinol” as used herein refers to the compounds of formula (1) (without any indication of the stereochemistry):

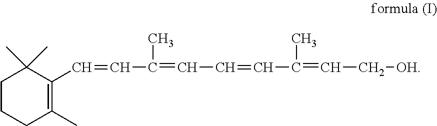 [SMILES: CC(C=CC1=C(C)CCCC1(C)C)=CC=CC(C)=CCO]

The cis/trans ratio as given herein refers to the wt.-% ratio of the sum of all cis-isomers to the respective all trans isomer of the respective retinoid, such as e.g. retinyl acetate, retinol, retinal or the like, as determined by known methods such as e.g. HPLC, assuming the same response factor for all isomers.

As used herein, a “retinol mixture” comprises retinol isomers, such as cis- and trans-isomers, in a ratio as defined herein.

As used herein, the cis/trans ratio, particularly the ratio of cis-isomers to trans-isomers in a retinol mixture, more particularly the ratio of cis-retinol to trans-retinol, means comparing the wt % of a cis-isomer to the wt % of the corresponding trans-isomer, all based on total retinoids in said mixture. Thus, a mixture comprising a cis/trans ratio of 0.01 with regards to retinol is a mixture with e.g. 1 wt % cis-retinol based on total retinoids within said mixture and 100 wt % trans-retinyl acetate based on total retinoids within said mixture.

As used herein, the term “trans-retinol” or the like are known to the skilled person and in line with the IUPAC-IUB nomenclature and mean that all double bonds in such retinol compound, including the compound according to formula (1), are in trans configuration. Thus, if one of several double bonds is not in the trans configuration but in the cis configuration, such isoforms are named herein as e.g. cis-retinol.

Retinol isomers in cis-configuration that are covered by the present invention include but are not limited to 9-cis-retinol, 11-cis retinol, 13-cis retinol, 9,13-di-cis-retinol, 11,13-di-cis retinol, 13-cis-3,4-didehydro retinol, 9-cis-3,4-didehydro retinol, 9,13-di-cis-3,4-didehydro retinol (for review, see e.g. Table 1 in Gundersen and Blomhoff, J. Chromatogr. A 935, 13-43, 2001).

In all embodiments of the present invention the cis/trans ratio of retinol isomers as defined above, i.e. wt % of cis- or trans-retinol, based on total retinoids in the mixture, is less than 0.01 (1:100), preferably a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003.

According to further inventive embodiments, the cis/trans ratio of retinol isomers as defined above, i.e. wt % of cis- or trans-retinol, based on total retinoids in the mixture, is 1:more than 100, preferably a cis/trans ratio of 1:105, 1:105.26, 1:110, 1:111.11, 1:117.65, 1:120, 1:125, 1:130, 1:133.33, 1:135, 1:140, 1:142.86, 1:150, 1:153.85, 1:155, 1:160, 1:165, 1:166.67, 1:170, 1:175, 1:180, 1:181.82, 1:185, 1:190, 1:195, 1:200, 1:204.08, 1:205, 1:210, 1:215, 1:220, 1:222.22, 1:225, 1:230, 1:232.56, 1:235, 1:240, 1:245, 1:250, 1:255, 1:260, 1:265, 1:270, 1:275, 1:280, 1:285, 1:285.71, 1:290, 1:300, 1:305, 1:310, 1:320, 1:325, 1:330, 1:333.33, 1:340, 1:345, 1:350, 1:357.14, 1:360, 1:370, 1:380, 1:390, 1:400, 1:420, 1:450, 1:454.55, 1:460, 1:480, 1:500, 1:520, 1:550, 1:555.56, 1:600, 1:666.67, 1:700, 1:800, 1:900, 1:1000, 1:1200, 1:1500, 1:2000, 1:2500, 1:2800, 1:4000, 1:5000, 1:7000 or less, such as 1:1000 or less, more preferably ranges of 1:101 to 1:10000, 1:125 to 1:1000, 1:140 to 1:500, 1:101 to 1:1000, 1:200 to 1:1000, 1:200 to 1:10000, 1:200 to 1:2000, 1:600 to 1:1000, 1:110 to 1:10000, 1:500 to 1:700, 1:150 to 1:400, 1:200 to 1:500, 1:250 to 1:400, 1:250 to 1:1000, most preferably a cis/trans ratio of 1:300 to 1:400, even more preferably a cis/trans ratio of about 1:333.

In a further embodiment, the present invention relates to the use of said mixture and/or said pharmaceutical, food, feed, household or cosmetic composition comprising said mixture of cis and trans isomers of retinol with a cis/trans ratio of less than 0.01 as defined herein as antimicrobial agent, i.e. an agent which exhibits an antimicrobial activity. In particular, the present invention is directed to the use of a mixture and/or pharmaceutical, food, feed, household or cosmetic composition comprising said mixture according to the present invention as anti-fungal agent, more particularly as an agent for inhibiting (reducing) and/or abolishing the growth of fungi such as in particular selected from Aspergillus, more particularly A. brasiliensis.

In another embodiment, the invention relates to a method or process for inhibiting and/or abolishing growth of microbial cells, in particular fungal cells, more particularly cells of Aspergillus, preferably A. brasiliensis, said method comprising contacting said microbial cells with a mixture and/or pharmaceutical, food, feed, household or cosmetic composition comprising said mixture according to the present invention.

In all embodiments of the present invention and as used herein, the term “antimicrobial activity” or “antimicrobial effect” preferably refers to the capability of inhibiting and/or abolishing the growth of microbes, particularly fungal cells, more particularly cells of Aspergillus, preferably A. brasiliensis, in a given sample or in a human or animal in need thereof. The terms “abolishing” and “killing” are used interchangeably herein.

Due to the antimicrobial activity against fungi, particularly Aspergillus, preferably A. brasiliensis, the mixtures and/or pharmaceutical, food, feed, household or cosmetic compositions comprising said mixtures according to the present invention are further suitable for preservation of food, cosmetic, pharmaceutical, medicinal or household products, plastics, paper and/or paints.

Thus, in a further embodiment the present invention relates to the use of a mixture and/or pharmaceutical, food, feed, household or cosmetic compositions comprising said mixtures according to the present invention for the preservation of food, cosmetic, pharmaceutical, medicinal, or household products, plastics, paper and/or paints as well as to protect food, cosmetic, pharmaceutical, medicinal or household products, plastics, paper and/or paints against mold, particularly against mold formation caused by Aspergillus, such as preferably A. brasiliensis.

The retinol mixture comprising cis and trans isomers of retinol and or pharmaceutical, food, feed, household or cosmetic compositions comprising said mixtures according to the present invention can either be prepared by admixing the respective all trans isomer with one or more cis isomers obtained by chemical or biological processes. Methods to prepare such all trans and/or cis isomers are well known to a person skilled in the art. Alternatively, the mixture can be prepared in said isomer ratio by adjusting the processes accordingly.

Advantageously, in all embodiments of the present invention, the retinol mixtures are biologically produced through a fermentation process, wherein trans-retinol produced by fermentation can be treated with heat to form cis-retinol, to achieve the proper levels mentioned in all the embodiments of the present invention (see e.g. McBee et al., JBC, Vol. 276, No. 51, pp. 48483-48493, 2001).

Advantageously, in all embodiments of the present invention, the retinol mixture comprising cis and trans isomers of retinol, with a ratio of cis-retinol to trans-retinol of less than 1:100 (i.e. a ratio of less than 0.01), particularly mixtures of retinol isomers with a cis/trans ratio of about 0.003, are biologically produced through a fermentation process, preferably leading to a retinol mixture with yellowish color.

As known in the art, color can be precisely described in several different co-ordinate systems, such as XYZ, RGB, CYMK, or L*a*b* (CIELAB according to EN ISO/CIE 11664-4: 2019). A preferred method is the definition via the L*a*b* system. The skilled person knows which instrument to use depending on the different color measurement systems and how to measure the color of the mixtures as described herein.

A “yellowish” color as used herein means a color as defined by the L*a*b* color system, particularly wherein L*a*b* being (3<L*<100, −25<a*<30, 10<b*<150), such as e.g. L* being in the range of 50 to 100, a* being in the range of −25 to 10 and b* being in the range of 40 to 150, preferably wherein L* being in the range of 80 to 100, a* being in the range of −22 to 1, b* being in the range of 40 to 85 or 100 to 140, more preferably L* being in the range of 80 to 95, a* being in the range of −17 to −1, b* being in the range of 105 to 135, particularly L* being about 80, 81, 82, 83, 84, 85, 86, 87, 88, 89, 90, 91, 92, 93, 94, 95, 96, 97, 98, 99, 100 and a* being about −24.9, −24, −23, −22, −21, −20, −19, −18, −17, −18, −17, −16, −15, −14, −13, −12, −11, −10, −9, −8, −7, −6, −5, −4, −3, −2, −1, 0 and b* being about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135.

In a particular embodiment, the retinol mixture comprising said mixture of cis and trans isomers of retinol as defined herein, are produced in a fermentation process using suitable retinol producing host cells, such as e.g. bacterial or fungal cells, (see e.g. Sun et al, ACS Synth. Biol. 2019 Sep. 20; 8(9):2131-2140; Jang et al., Microbial Cell Factories 2011, 10:59), wherein the cells are expressing the respective enzymes, such as e.g. trans-selective enzymes, i.e. beta-carotene oxidase (BCOs) involved in biosynthesis of retinol from conversion of beta-carotene into retinal, that might be further enzymatically converted into retinol.

Particularly, the fermentation is fed ethanol, corn sugar or corn oil all derived from agricultural production. The fermentation products comprising retinyl esters, particularly retinyl acetate, might be extracted in an aliphatic phase and subsequently purified to crystalline forms by methods as used in the art.

The term “biologically produced” oas used herein means that the retinol is produced by the help of biotechnological process, such as a fermentation process including cultivation of a suitable (carotenoid and/or retinoid producing) host cell expressing the respective enzymes involved in conversion of a suitable carbon source into retinal and furthermore into retinol as defined herein, wherein the host cell might be selected from bacteria, fungi, particularly yeast, plant or algae, particularly from fungal cells, such as e.g. Yarrowia or Saccharomyces, or bacterial cells, such as e.g. E. coli. It might furthermore be selected from other suitable microorganism known to produce retinol. “Bio-produced”, “biologically-derived” and “biologically produced” or “bio-based” are used synonymously herein. Accordingly, said biologically produced retinoids comprising retinol are composed of carbon from atmospheric carbon dioxide (also referred to as carbon of atmospheric origin) converted to sugars and starches by green plants and thus preferably sugars and starches composed of non-fossil-fuel carbon. It also includes the use of isolated and/or immobilized enzymes in a process for generation of the retinol mixture as defined herein, such as specific enzymes capable of selectively catalyzing the formation of the specific trans/cis ratio of retinol in a mixture as defined herein.

In one aspect, the retinol mixture as defined herein is produced via fermentation using a fungal host cell, such as e.g. an oleaginous yeast, particularly Yarrowia, said host cell being cultivated in the presence of a suitable carbon source and in the presence of a plant-based second phase, e.g. vegetable oil, such as e.g. corn oil, as second phase solvent, wherein the retinoids comprising retinol are accumulated in the second phase.

Suitable plant-based second phase solvents might be selected from any vegetable oil, including but not limited to oleic, palmitic, steric or linoleic acid and glycerol, such as e.g. corn, olive, cottonseed, rapeseed, sesame, canola, safflower, sunflower, soybean, grapeseed, or peanut oil, preferably corn oil.

Carbon sources to be used for the present invention might be selected from linear alkanes, free fatty acids, ethanol, glucose and/or mixtures thereof.

As used herein, a “bio-based” compound has a C-14/C-12 isotope ratio in the range of from 1:0 to greater than 0:1, as in contrast to a fossil-based compound with a C-14/C-12 isotope ratio of 0:1. Bio-based content of a compound can be measured by known radiocarbon and isotope ratio mass spectrometry analysis or accelerator mass spectrometry, such as e.g. the ASTM test method D6866 such as D6866-05 or D6866-20, DIN SPEC 91236 (CEN/TS 16137), ISO 16620, or DIN EN 16785/1, wherein the C-14/C-12 isotope ratio in a sample is measured and compared to a standard 100% biobased material to give percent biobased content of the sample.

“Carbon of atmospheric origin” as used herein refers to carbon atoms from carbon dioxide molecules that have recently, in the last few decades, been free in the earth's atmosphere. Such carbons in mass are identifiable by the presence of particular radioisotopes as described herein. “Green carbon”, “atmospheric carbon”, “environmentally friendly carbon”, “life-cycle carbon”, “non-fossil fuel-based carbon”, “non-petroleum based carbon”, “carbon of atmospheric origin”, and “biobased carbon” are used interchangeably herein.

In all embodiments of the present invention, advantageously the retinol mixture is produced by merely organic, renewable, bio-based feedstock, particularly fermentatively produced, as such retinol has an anthropogenic CO₂ emission profile of zero upon biodegradation because all of the CO₂ molecules released during degradation from such “fermentatively-derived” or “fermentatively-produced” retinol have an atmospheric origin. Thus, the net release of CO₂ to the atmosphere is zero.

Thus, the present invention is particularly directed to a mixture comprising retinol comprising cis- and trans-isomers as defined herein, with a cis-/trans ratio (defined as wt % based on total retinoids in the mixture) of less than 0.01, wherein said mixture has at least about 20%, such as 25, 30, 35, 40, 45, 50, 55, 60, 65, 70, 75, 80, 85, 90, 95, 98 or 100% biobased carbon according to the definition given in the present application.

Particularly, the present invention is directed to a biodegradable composition comprising a mixture of retinyl acetate with a cis-/trans-ratio of less than 0.01 as defined herein, wherein the retinyl acetate mixture in said composition has an anthropogenic CO₂ emission profile of zero upon biodegradation.

In one preferred embodiment the retinol mixture according to the present invention consists essentially of cis and trans isomers of retinol in the ranges and with all the definitions and preferences as given herein.

The term “consists essentially of” as used according to the present invention means that the total amount of ingredients within the mixture ideally sum up to 100 wt.-%. It is however not excluded that small amounts of impurities or additives may be present in the mixture, with the proviso that the total amount of such impurities is preferably less than about 5 wt.-%, more preferably less than about 3, 2.1 wt.-% and which are e.g. introduced via the respective raw materials and/or processes used.

As used herein, the term “impurities” and “additives” are used interchangeably herein and refer to co-ingredients within the inventive mixture and that are present in the inventive mixture in an amount of less than 5 wt.-% based on all ingredients present in the mixture.

If the retinol mixture according to the present invention consists essentially of retinol with a cis/trans ratio as defined herein, then the purity of said mixture, i.e. the (total) amount of cis and trans retinol, is preferably at least about 95%, more preferably at least about 96, 97, 98%, most preferably at least about 98%, as determined by known methods such as e.g. HPLC, particularly reversed phase C4 HPLC.

As used herein, the term “purity” means the sum of cis- and trans isomers in relation to the total weight, e.g. the sum of cis-retinol and trans-retinol according to the definition given herein gives a percentage of purity based on total retinoids set to 100% purity. Thus, if a mixture consists essentially of 98.03 wt % trans-retinol and 0.25 wt % cis-retinol, said mixture has a purity with regards to retinol of 98.28%. Preferably, and in accordance with all embodiments of the present invention, the retinoid mixture comprises less than 0.3 wt % cis-retinol, based on total retinoids in said mixture.

In some embodiment, the mixtures comprising cis and trans retinol with a cis/trans ratio as defined herein further comprise small amounts of additives such as e.g. dihydro-retinoids, including dihydro-retinol and/or dihydro-retinyl acetate, particularly in a range of 0.2 to 0.01 wt % or less, such as e.g. 0.2, 0.18, 0.16, 0.15, 0.14, 0.12, 0.1, 0.05, 0.01 wt % or less, preferably a range of 0.2 to 0.1, 0.17 to 0.06, 0.1 to 0.05, 0.04 to 0.01 wt %, more preferably a percentage of 0.1 wt % or less, all based on total retinoids. Also preferably, the percentage of dihydro-retinyl acetate is about 0.05 wt % or less, particularly about 0.01 wt % or less based on total retinoids within said mixture.

To make use of the antimicrobial activity against A. brasiliensis yeast of the retinol mixtures they can be used in a multiplicity of applications, such as, for example, cosmetic, pharmaceutical, nutritional, medicinal or household products, plastics, paper and/or paints.

In all embodiments of the present invention the amount of cis and trans retinol (based on the active, i.e. the retinol) in the food, cosmetic, pharmaceutical, medicinal, or household products, plastics, paper and/or paints is preferably selected from 0.005 to 2 wt.-%, preferably from 0.01 to 1 wt.-%, more preferably from 0.03 to 0.75 wt.-% and most preferably from 0.05 to 0.5 wt.-%, based on the total weight of the respective product, plastic, plastics, paper and/or paint.

In a particular embodiment, the invention relates to a method of preventing microbial decay and breakdown of food, cosmetic, pharmaceutical, medicinal, or household products, plastics, paper and/or paints, wherein said method comprises adding to said products, plastics, papers and/or paints a mixture and/or pharmaceutical, food, feed, household or cosmetic composition comprising said mixture according to the present invention (based on the active, i.e. the retinol) in an amount of 0.005 to 2 wt.-%, more preferably in the range of 0.01 to 1 wt.-%, most preferably in the range of 0.03 to 0.75 wt.-% such as in the range of 0.05 to 0.5 wt.-% as an antimicrobial agent. In a particular embodiment, the method also encompasses the step of appreciating the result.

“Prevention” includes not only total prevention, delaying the onset of, lessening the severity of an incident/symptoms of the condition, and/or increasing the length of time between recurrence of the incidence/condition.

In a further embodiment, the invention also relates to a method of preserving food, cosmetic, pharmaceutical, medicinal, or household products, plastics, paper and/or paints against microbial contamination or growth, wherein said method comprises the addition of a retinol mixture and/or pharmaceutical, food, feed, household or cosmetic composition comprising said mixture according to the present invention (based on the active, i.e. the retinol) in an amount of 0.005 to 2 wt.-%, more preferably in the range of 0.01 to 1 wt.-%, most preferably in the range of 0.03 to 0.75 wt.-% such as in the range of 0.05 to 0.5 wt.-% to said product, plastic, plastisol, paper and/or paint as an antimicrobial agent.

In a particular advantageous embodiment, the invention relates to a method of preventing microbial decay and breakdown of food, cosmetic, pharmaceutical, medicinal, or household products, plastics, paper and/or paints furthermore comprising water and optionally at least one further agent selected from the group consisting of surfactants, emulsifiers, thickeners as they are particular susceptible to microbial decay and breakdown.

Preferred products in all embodiments of the present invention are pharmaceutical, food, feed, household or cosmetic compositions comprising the mixtures. Said compositions preferably comprise water and at least one further agent selected from the group consisting of surfactants, emulsifiers, thickeners.

Thus, in another embodiment, the invention is also directed to cosmetic or pharmaceutical products comprising water and at least one agent selected from the group consisting of surfactants, emulsifiers, thickeners and oils, wherein the pharmaceutical, food, feed, household or cosmetic composition furthermore comprises a retinol mixture according to the present invention (based on the active, i.e. the retinol) in an amount of 0.005 to 2 wt.-%, more preferably in the range of about 0.01 to 1 wt.-%, most preferably in the range of about 0.03 to 0.75 wt.-% such as in the range of 0.05 to 0.5 wt.-%, based on the total weight of the product.

It is furthermore advantageous, that the water content in said cosmetic or pharmaceutical products according to the present invention is selected in the range from 30 to 90 wt.-%, from 40 to 90 wt.-%, from 45 to 90 wt.-% or from 50 to 90 wt.-%, based on the total weight of the composition. Further suitable ranges are from 30 to 75 wt.-%, from 30 to 70 wt.-%, from 30 to 60 wt.-% and from 40 to 60 wt.-%.

Advantageously, the retinol mixture and/or pharmaceutical, food, feed, household or cosmetic composition comprising said mixture including products comprising said compositions according to the present invention can also be used in combination with traditional preservatives to improve the preservative activity thereof.

It is well understood that the use according to the invention is preferably non-therapeutic.

The cosmetic or pharmaceutical products according to the present invention are in particular topically applied to mammalian keratinous tissue such as in particular to human or animal skin or the human or animal scalp and hair.

Thus, in one embodiment the present invention is directed to a retinol mixture and/or pharmaceutical or cosmetic composition comprising said mixture as defined herein, for the use as topical product, i.e. cosmetic product topically applied to the human or animal keratinous tissues including skin, scalp and hair.

The term “cosmetic product” as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

Suitable surfactants, emulsifiers, thickeners, and oils for the purpose of the present inventions are all surfactants, emulsifiers, thickeners, and oils commonly used in cosmetic applications and which are e.g. listed in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The products according to the present invention are generally prepared by admixing a mixture including formulation I or II according to the present invention in an amount selected in the range 0.005 to 2 wt.-%, more preferably in the range of about 0.01 to 1 wt.-%, most preferably in the range of about 0.03 to 0.75 wt.-% such as in the range of 0.05 to 0.5 wt.-%, based on the total weight of the product with a suitable carrier.

The cosmetic or pharmaceutical products according to the present invention preferably further comprise a physiologically acceptable medium, that is to say a medium compatible with keratinous substances, such as the skin, mucosa, and keratinous fibers. In particular, the physiologically acceptable medium is a cosmetically or pharmaceutically acceptable carrier.

The term “cosmetically or “pharmaceutically acceptable carrier” refers to all carriers and/or excipients and/or diluents conventionally used in cosmetic compositions or pharmaceutical compositions. As used herein, it can be understood to be a physiologically acceptable medium, i.e. a medium compatible for the intended use such as e.g. with keratinous substances, such as the skin, mucous membranes, and keratinous fibers.

The cosmetic compositions or products of the present invention preferably comprise from 50% to 99.995%, such as 50 to 99.95%, more preferably from 60% to 99.995%, such as 60 to 99.95%, still more preferably from 75% to 99%, and most preferably, from 80% to 98% such as 90% to 98%, by weight of the composition, of a carrier, based on the total weight of the composition.

In a particular advantageous embodiment, the carrier consists furthermore of at least about 30 wt.-%, more preferably of at least about 40 wt.-%, most preferably of at least about 45, 50, 60, 70, 80, 90 wt.-% of water, such as in particular a range of from 30 to 90 wt.-%, such as from 40 to 90 wt.-%, from 45 to 90 wt.-% or from 50 to 90 wt.-%, based on the total weight of the composition. Further suitable ranges are from 30 to 75 wt.-%, from 30 to 70 wt.-%, from 30 to 60 wt.-% and from 40 to 60 wt.-%.

Preferably, the cosmetic or pharmaceutical products according to the invention are in the form of a suspension or dispersion in solvents or fatty substances, or alternatively in the form of an emulsion or micro emulsion (in particular of O/W- or W/O-type), PIT-emulsion, nano emulsion, multiple emulsion (e. g. O/W/O- or W/O/W-type), pickering emulsion, hydrogel, lipogel, one- or multiphase solution or vesicular dispersion.

The cosmetic or pharmaceutical products in accordance with the invention can be in the form of a liquid, lotion, a thickened lotion, a gel, a cream, a milk, an ointment or a paste.

The cosmetic or pharmaceutical products according to the invention have a pH in the range of 3-10, preferably in the range of pH of 3-8, most preferred in the range of pH 3.5-7.5. The pH is adjusted by methods known to a person skilled in the art, e.g. by using an acid such as a hydroxy acid including glycolic acid, lactic acid, malic acid, citric acid and tartaric acid or a base such as e.g. sodium or potassium hydroxide or ammonium hydroxide as well as mixtures thereof.

The amount of the cosmetic composition or product to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

Preferably, in the product according to the invention said acid, if present, is used in an amount of at lowest about 0.0001 wt.-%, such as e.g. in an amount of about 0.01 to 1 wt.-%, in particular in an amount of about 0.01 to 0.5 wt.-%.

The cosmetic products according to the present invention are in particular skin care preparations, functional preparations and/or hair care preparations such as most in particularly skin or hair care preparations.

Thus, in one embodiment the present invention is directed to the use of a retinol mixture and/or food, feed, household composition comprising said mixture as defined herein as skin or hair care preparations, i.e. for the preparation of cosmetical products applied to skin or hair of humans or animals.

Examples of skin care preparations are, in particular, light protective preparations (sun care preparations), anti-ageing preparations, preparations for the treatment of photo-ageing, body oils, body lotions, body gels, treatment creams, skin protection ointments, moisturizing preparations such as moisturizing gels or moisturizing sprays, face and/or body moisturizers, as well as skin lightening preparations.

Preferably in all embodiments of the present invention the skin care preparation is a deodorant, an anti-perspirant, or an anti-acne composition.

Examples of functional preparations are cosmetic compositions containing active ingredients such as hormone preparations, vitamin preparations, vegetable extract preparations, anti-ageing preparations, and/or antimicrobial (antibacterial or antifungal) preparations without being limited thereto.

Examples of hair care preparations which are suitable according to the invention and which may be mentioned are shampoos, hair conditioners (also referred to as hair rinses), hairdressing compositions, hair tonics, hair regenerating compositions, hair lotions, water wave lotions, hair sprays, hair creams, hair gels, hair oils, hair pomades or hair brilliantines. Accordingly, these are always preparations which are applied to the hair and the scalp for a shorter or longer time depending on the actual purpose for which they are used.

If the hair care preparations according to the invention are supplied as shampoos, these can be clear liquids, opaque liquids (with pearly luster effect), in cream form, gel-like or else in powder form or in tablet form, and as aerosols. The surfactant raw materials on which these shampoos are based can be anionic, cationic, nonionic and amphoteric in nature and also be present in combinations of these substances.

In another preferred embodiment, the cosmetic compositions according to the present invention are O/W emulsions, W/O emulsions and/or gels such as shower gels or hair gels.

The following examples are provided to further illustrate the compositions and effects of the present invention. These examples are illustrative only and are not intended to limit the scope of the invention in anyway.

The following embodiments (1) to (14) are particularly within the scope of the present invention:
(1): A mixture of cis and trans isomers of retinol, wherein the cis/trans ratio within said mixture is less than 0.01, a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003.
(2): A composition comprising the mixture of cis and trans-retinol of embodiment (1).
(3): Mixture or embodiment (1) or composition of embodiment (2), wherein the retinol is biologically-derived.
(4): Mixture or composition of embodiments (1), (2) or (3), wherein the mixture consists of at least about 95% retinol based on all components in the mixture.
(5): Use of a mixture or composition of embodiments (1), (2), (3) or (4) as an anti-fungal agent.
(6): The use of embodiment (5), wherein the antifungal is an agent that inhibits the growth of Aspergillus, preferably Aspergillus brasiliensis.
(7): The use of embodiment (5) or (6) in food, cosmetic, pharmaceutical or household products, plastics, paper and/or paints.
(8): The use of embodiments (5), (6) or (7) for improving preservation.
(9): A method or process of preventing microbial decay and breakdown of food, cosmetic, pharmaceutical and/or household products, plastics, paper and/or paints, wherein said method comprises adding to the products, plastics, paper and/or paints a mixture or composition of embodiments (1), (2), (3) or (4) as an antimicrobial agent in an amount in the range of 0.005 to 2 wt.-%, based on the total weight of the respective product, plastic, paper and/or paint.
(10): The method or process of embodiment (9), wherein the amount of the added mixture is in the range of 0.01 to 1 wt.-%, based on the total weight of the respective product, plastic, paper and/or paint.
(11): The use, method or process of embodiment (9) or (10), wherein the product furthermore comprises water and optionally at least one agent selected from the group consisting of surfactants, emulsifiers, thickeners, oils, and mixtures thereof.
(12): The use, method or process of embodiment (9), (10) or (11), wherein the product is a cosmetic composition in the form of a shampoo preparation, a hair conditioner, an O/W emulsion, a W/O emulsion or a gel.
(13): A method or process for abolishing and/or inhibiting growth of microbial cells, in particular fungal cells, said method comprising contacting said microbial cells with a mixture or composition of embodiments (1), (2), (3) or (4), particularly wherein the fungal cells are selected from Aspergillus, preferably Aspergillus brasiliensis.

### EXAMPLES

### Example 1: Antimicrobial Efficacy

Retinoid mixture comprising retinol as defined herein can be produced as described in e.g. Sun et al. (ACS Synth. Biol, 10.2021/acssynbio.9b00217), wherein the retinol are accumulated in the lipid phase with subsequent isolation/extraction with ethanol or the like as known in the art.

The antimicrobial efficacy of retinol having different cis/trans ratio has been assessed in analogy to the regulatory challenge test method (NF EN IS011930).

Thus, solutions of the respective retinol (reference retinol and inventive retinol mixture with purity of about 98%) in physiological serum with 0.85 wt.-% NaCl were prepared under sterile conditions, with a cis/trans retinol ratio of 0.01 for the reference and a ratio cis/trans retinol ratio of 0.003 for the inventive retinol mixture (Table 1). The presence of cis and trans-isomers were calculated by reversed phase C4 HPLC analysis.

TABLE 1
cis/trans ratio of tested samples, wherein “reference retinol” is
used as a control and “Retinol mix” refers to the retinol mixture
with the inventive cis/trans ratio. For further details see text.

| | Reference | |
|---|---|---|
| | retinol | Retinol mix |
| ratio cis/trans | 0.01 | 0.003 |

The solutions of the active(s) were then deposed in 96-deep well plates (1.6 ml/well). The wells were contaminated with A. brasiliensis to obtain the initial contamination as outlined in Table 2 below. After contamination, each well was thoroughly mixed to ensure a homogeneous distribution of the microbes. Then each plate was incubated at 22° C. for 24 h. The counting of the (remaining) population was carried out 24 h after contamination.

As can be seen in Table 2, the specific cis/trans mixture of retinol according to the present invention has an excellent antimicrobial effect against A. brasiliensis with reduction in the range of 30% (“A R”) as compared to the antimicrobial activity of the reference. The antimicrobial efficacy of commercially available trans-retinol (e.g. Retinol GS 50 from DSM or Retinol 50 C from BASF) is in the same range as the reference sample (not shown).

TABLE 2
contamination of samples according to Table 1 with A. brasiliensis.
The concentration of tested retinyl acetate with cis/trans ratio
as indicated in Table 1 was 0.25%. For further details see text.

| Inoculum | Reference | Retinol mixture | ΔR |
|---|---|---|---|
| colony counts [cfu/ml] | | | [%] |
| 100000 | 1000 | 700 | −30 |

### Example 2: Formulations

Cosmetic formulations in the form of gels, emulsions, shampoo, hair cream, lotion, make-up, toner, after-shave, creams, hand sanitizer are created according to methods known in the art. If not indicated otherwise the pH of the formulations is adjusted to pH 3-7.5 as deemed appropriate. In the following formulations the term “NEW INGREDIENT” refers to retinol (Retinol mix) with a cis/trans ratio as indicated in Table 1.

TABLE 3
Retinol comprising gels. For further details see text.

| | 1 | 2 | 3 | 4 | 5 |
|---|---|---|---|---|---|
| Example: Gels | Wt.-% | | | | |
| Acrylates/Octylacrylamide Copolymer | 1 | 1 | 1 | 1 | 1 |
| Alcohol Denat. | 10 | 30 | 59 | 5 | 54 |
| Butylene Glycol Dicaprylate/Dicaprate | | | 2 | 4 | |
| C₁₂₋₁₅ Alkyl Benzoate | 8.5 | 7.5 | | 2 | 5 |
| Phenylethylbenzoate | | | | 2.5 | |
| Cocoglyceride | | | 2 | 5 | |
| Tridecylsalicylate | 1.5 | 3 | 1 | | 3 |
| Hydroxypropylcellulose | 0.8 | 0.5 | 0.8 | 0.45 | 0.5 |
| Butyl Methoxydibenzoylmethane | | 2.5 | 4.5 | | 3 |
| 2.4.6-Tribiphenyl-4-yl-1.3.5 Triazine | | | 6 | | |
| Diethylamino Hydroxybenzoyl Hexyl | | 1.0 | | | |
| Benzoate | | | | | |
| Ethylhexyl Methoxycinnamate | | 6.5 | | | |
| Ethylhexyl Salicylate | | | | | 5 |
| Homosalate | | | 4.5 | | |
| Octocrylene | | 3.8 | | | 4 |
| Ethylhexyl Triazone | | 1 | | | |
| Drometrizole Trisiloxane | | 1 | | | 1 |
| NEW INGREDIENT | 0.5 | 0.3 | 0.1 | 0.05 | 0.15 |
| Bis-Ethylhexyloxyphenol | | | 1 | | |
| Methoxyphenyltriazine | | | | | |
| Vitamin E Acetate | | | 0.2 | 0.5 | |
| Niacinamide | | 2.0 | | | 3.0 |
| Glycerin | 8 | 5 | 4 | 6 | 2 |
| Ascorbylglucoside | 0.1 | | | 1.0 | |

| | |
|---|---|
| Fragrance. Colours | q.s. |
| Water | ad 100 |

TABLE 4
Retinol comprising O/W emulsions. Palmitoyl Oligopeptide & Palmitoyl
Tetrapeptide-7 are given in [ppm]. For further details see text.

| | 1 | 2 | 3 | 4 | 5 | 6 | 7 |
|---|---|---|---|---|---|---|---|
| Example: O/W Emulsions | Wt.-% | | | | | | |
| Glyceryl Stearate Citrate | 2 | 2 | 3 | | | | |
| Glyceryl Stearate SE | | | | 1 | 1 | 1.5 | |
| Cetearyl Alcohol + PEG-40 Rizinusoil + | | | | 2.5 | 2.5 | 3 | |
| Sodium Cetearyl Sulfate | | | | | | | |
| Potassium Cetyl Phosphate | | 0.5 | | | | | 2 |
| Cetearyl Alcohol | | | 1 | 1 | | | 2 |
| Stearyl Alcohol | 0.5 | | | | | 2 | |
| Coco caprylate/caprate | 1 | 1 | | | 3 | | |
| Acrylates/C₁₀₋₃₀ Alkyl Acrylate Crosspolymer | 0.1 | 0.2 | | | 0.1 | | |
| Carbomer | | 0.2 | 0.3 | 0.1 | | | |
| Xanthan Gum | 0.4 | | 0.2 | 0.2 | 0.3 | 0.4 | 0.2 |
| C₁₂₋₁₅ Alkyl Benzoate | | 3 | | | 5 | | 4 |
| 2-Phenylethylbenzoate | 5 | 2 | | | | | |
| Butylene Glycol Dicaprylate/Dicaprate | 5 | | | | 3 | 3 | |
| Tridecylsalicylate | | 2.5 | | 3 | | 15 | 5.9 |
| Dicaprylcaprate | 2 | 2 | | | 2 | 2 | |
| Cyclomethicone | | | | 5 | 10 | | 5 |
| Dimethicone | | | | | 5 | | |
| PVP Hexadecene Copolymer | | 0.5 | | | | 1 | 2 |
| Propylene Glycol | | | 1 | | 5 | 3 | |
| Ascorbylglucoside | | 0.1 | | | 0.5 | | |
| Niacinamide | 1 | | | | 2 | | |
| Tranexamic Acid | | | 3 | 0.5 | | 1.5 | |
| Glycerin | 3 | 3 | 7 | 5 | 13 | 3 | |
| Propanediol | | 2 | | 5 | | | 3 |
| Alcohol denat. | 2 | 3 | | 7 | | | |
| Titanium Dioxide | 3 | | | 2 | | | 3 |
| Phenylene-1.4-bis-(2-benzimidazyl)- | 3 | 2 | | | | | |
| 3.3-5.5-tetrasulfonic Acid | | | | | | | |
| Ethylhexyl Triazone | 2.5 | 2 | | 1 | | | 1 |
| Phenylbenzimidazole Sulfonic Acid | | | 2 | | | 1 | 2 |
| 4-Methoxycinnamate (2-ethylhexyl) ester | | 5 | | 2 | | | |
| Butyl Methoxydibenzoylmethane | 5 | 1 | | | 3 | | 4 |
| Ethylhexylsalicylate | 5 | | 0.5 | 4 | 5 | | 4 |
| Polysilicone-15 | | | 4 | | | | 1 |
| Isoamyl p-Methoxycinnamate | | 3 | 6 | | | | |
| Diethylamino Hydroxybenzoyl Hexyl Benzoate | | | | 6 | | | |
| Methylene Bis-Benztriazoyl Tetramethylbutylphenol | | | 5 | | | | |
| 1.1′-(1.4-piperazinediyl)bis [1-[2-[4-(diethylamino)- | | | 2 | | | | |
| 2-hydroxybenzoyl] phenyl]-methanone | | | | | | | |
| Octocrylene | 3 | | | | 5 | 7 | 4 |
| Bis-Ethylhexyloxyphenol Methoxyphenyltriazine | | 1 | | 1 | | 1 | 0.5 |
| NEW INGREDIENT | 0.1 | 0.2 | 0.15 | 0.3 | 0.7 | 0.5 | 0.25 |
| Butyloctyl Methoxycrylene | | | | | 3 | | |
| Diethylhexyl Syringylidenemalonate | | | | | | | 2 |
| Vitamin E Acetate | 0.2 | 0.2 | 0.2 | 0.3 | 0.1 | 0.5 | 0.3 |
| Palmitoyl Oligopeptide & Palmitoyl Tetrapeptide-7 | | 10 | | | 30 | | |
| Disodium EDTA | 0.1 | 0.1 | 0.2 | 0.2 | 0.5 | 0.2 | 0.2 |
| DHA | | | | | 2 | | |
| Erythrulose | | | | 1.9 | | | |
| Silica | 1 | | 2.5 | | | 0.5 | |
| Methyl Methacrylate Crosspolymer | | | | 1 | | | 1 |
| Silica & Methicone | | 2 | | | 3 | | |

| | |
|---|---|
| Fragrance. Preservation agents | q.s |
| Colours. etc. | q.s |
| Citric Acid. Sodium Citrate | q.s |
| Sodium Hydroxide | q.s |
| Tromethamine | q.s |
| Water | ad 100 |

TABLE 5
Retinol comprising O/W emulsions. Palmitoyl Pentapeptide-4 is
given in [ppm]. For further details see text.

| | 1 | 2 | 3 | 4 | 5 |
|---|---|---|---|---|---|
| Example: O/W Emulsions | Wt.-% | | | | |
| Glyceryl Stearate | 2.5 | 2 | 1.2 | 1 | |
| PEG-40 Stearate | 1 | | | | |
| PEG-100 Stearate | | 2.5 | | | |
| Ceteareth-20 | | | | | 1 |
| Potassium Cetyl Phosphate | | 0.5 | | | 1 |
| Stearic Acid | | | 2.5 | 3 | |
| Cetearyl Alcohol | 4 | | | 2 | |
| Stearyl Alcohol | | 2 | 1 | | |
| Cetyl Alcohol | | | 1 | 1 | |
| Acrylates/C₁₀₋₃₀ Alkyl Acrylate | | | | 0.2 | 0.2 |
| Crosspolymer | | | | | |
| Carbomer | 0.1 | | 0.2 | | |
| Xanthan Gum | | 0.3 | | | |
| C₁₂₋₁₅ Alkyl Benzoate | 5 | | | 2 | 5 |
| Petrolatum | 5 | | 3 | | |
| Butylene Glycol Dicaprylate/Dicaprate | | 4 | 2 | | 9 |
| Hydrogenated Polydecene | | | 3 | | 2 |
| Caprylic/Capric Triglyceride | 1 | 3 | | 5 | |
| Cyclomethicone | | 5 | 2 | | |
| Methylpropandiol | 2 | | | | 3 |
| Glycerin | 4 | 4 | 3 | | 3 |
| Propanediol | | 3 | | 3 | |
| Glyceryl Glucoside | | 0.5 | 1 | | |
| Alcohol denat. | | 3 | 0.5 | 10 | |
| Butylene Glycol | | | 3 | | |
| Ascorbylglucoside | | 0.5 | | 1.0 | |
| Ubiquinone (Coenzyme 10) | 0.1 | | 0.05 | | |
| Hyaluronic acid | | | | | 0.2 |
| Bisabolol | 0.5 | | | | |
| Palmitoyl Pentapeptide-4 | | | 20 | | |
| Isotridecylsalicylate | | | 1 | 3 | 5 |
| Titanium Dioxide | 1 | | 0.5 | 2 | |
| 2.4.6-Tribiphenyl-4-yl-1.3.5 Triazine | | | 0.5 | 4 | |
| Ethylhexyl Bis- | | 1 | | | 0.5 |
| Isopentylbenzoxazolylphenyl Melamine | | | | | |
| Ethylhexyl methoxycinnamate | | | | | 2 |
| Phenylbenzimidazole Sulfonic Acid | 1.5 | | | 2 | |
| Butyl Methoxydibenzoylmethane | 2.5 | 1 | | 2 | 2 |
| Methylbenzylidene Camphor | | | | | 2 |
| Disodium Phenyl Dibenzimidazole | | | 1 | | |
| Tetrasulfonic Acid | | | | | |
| NEW INGREDIENT | 0.1 | 0.15 | 0.25 | 0.3 | 0.5 |
| Octocrylene | | 5 | | | |
| Polysilicone-15 | | | | 2 | |
| Ethylhexyl Salicylate | 3 | | | | 5 |
| Homosalate | | | 4 | | 2 |
| Benzophenone-3 (Oxybenzone) | | 2 | | | |
| Drometrizole Trisiloxane | 0.5 | | | 1 | |
| Terephthalidene Dicamphor Sulfonic | 1.5 | | | 0.5 | |
| Acid | | | | | |
| Butyloctyl Methoxycrylene | | 1 | | | |
| Silica | 1 | | 2.5 | | |
| Silica & Methicone | | 4 | | 1 | 2.5 |
| Methyl Methacrylate Crosspolymer | | | | 1 | |

| | |
|---|---|
| Disodium EDTA | 0.1 |
| Fragrance. Preservatives | q.s |
| Sodium Hydroxide | q.s |
| Water | Ad 100 |

TABLE 6
Retinol comprising W/O emulsions. Palmitoyl Tripeptide-
38 is given in [ppm]. For further details see text.

| | 1 | 2 | 3 | 4 | 5 |
|---|---|---|---|---|---|
| Example: W/O Emulsions | Wt.-% | | | | |
| Polyglyceryl-2 Dipoly-hydroxystearate | 3 | 5 | 3 | | |
| PEG-30 Dipoly-hydroxystearate | | | 2 | 3 | 4 |
| Sodium Starch Octenylsuccinate | 0.5 | 0.4 | 0.6 | 0.3 | 0.5 |
| Glycine | | | 0.5 | 0.4 | |
| Alcohol denat. | | 5 | 2 | | 8 |
| Magnesium Sulfate | 0.2 | 0.3 | 0.3 | 0.4 | 0.5 |
| C₁₂₋₁₅ Alkyl Benzoate | 5 | 3 | | | 5 |
| Triheptanoin | | 2 | | | |
| Butyleneglycol Dicaprylat/Dicaprate | 5 | | | | 3 |
| Dicaprylyl Ether | | | | | 2 |
| Mineral Oil | | 4 | | 6 | |
| Octyldodecanol | 2 | | | | |
| Dicapryl Caprate | | 2 | | | 2 |
| Cyclomethicone | 5 | | 5 | 10 | |
| Dimethicone | | | | 5 | |
| Isohexadecane | | 1 | | | |
| Butylene Glycol | 5 | 8 | | | |
| Glycerin | | 5 | 7 | 5 | 3 |
| Propanediol | 3 | | | 5 | |
| Tridecylsalicylate | | 1 | 2 | | |
| 2-Phenylethyl-benzoate | | | 2 | | 4 |
| Isopropyl Lauroyl Sarcosinate | | | 1 | | 2 |
| Ethylhexylmethoxycinnamate | 2 | | | | |
| Ethylhexyl Triazone | 2 | 3 | 3 | | |
| Ethylhexyl Bis-Isopentyl- | | | | 3 | |
| benzoxazolylphenyl Melamine | | | | | |
| Diethylhexyl Butamido Triazone | | 1.5 | 1.5 | | |
| Butyl Methoxy dibenzoylmethane | 1 | 4 | 3 | | 5 |
| Methylbenzylidene Camphor | | | | | 1 |
| 2.4.6-Tribiphenyl-4-yl-1.3.5 Triazine | | | 5 | | |
| 2-(4-Diethylamino-2-hydroxybenzoyl)- | | | | 2 | |
| Benzoic Acid Hexylester | | | | | |
| NEW INGREDIENT | 0.2 | 0.5 | 0.1 | 0.2 | 0.3 |
| Palmitoyl Tripeptide-38 | | 5 | | | |
| Titanium Dioxide (Parsol TX) | 5 | | 2 | | 3 |
| Polysilicone-15 | 2 | | | | 1.5 |
| Octocrylene | | 3.6 | | | |
| Ethylhexyl Salicylate | | | | | 5 |
| Phenylbenzimidazole Sulfonic Acid | 3 | | | | |
| Bis-Ethylhexyl-oxyphenol Methoxy- | 1 | 2 | 2 | | 2 |
| phenyltriazine | | | | | |
| Methylene Bis-benzotriazolyl | 2 | | 3 | | 1 |
| tetramethylbutylphenol | | | | | |
| 1.1′-(1.4-piper-azinediyl)bis[1-[2-[4- | | | | 2 | |
| (diethylamino)-2-hydroxybenzoyl] | | | | | |
| phenyl]-methanone | | | | | |
| Butyloctyl Methoxycrylene | | 4 | | | |
| Vitamin E Acetate | 0.2 | 0.2 | 0.2 | 0.3 | 0.1 |
| Ascorbylglucoside | 0.2 | | 0.5 | | |
| Disodium EDTA | 0.1 | 0.1 | 0.2 | 0.2 | 0.2 |

| | |
|---|---|
| Fragrance. Preservatives | q.s |
| Water | ad 100 |

TABLE 7
Retinol comprising O/W emulsion for facial cream. For
further details see text.

| | 1 | 2 |
|---|---|---|
| Example: O/W-Emulsion: facial cream | Wt.-% | |
| Polyglyceryl-3-Methylglucosedistearate | 2.0 | 2.0 |
| Sorbitanstearate | 3.0 | 3.0 |
| Cetylalcohol | 2.0 | 2.0 |
| Myristylmyristate | 1.0 | 1.0 |
| Dicaprylylether | 3.0 | 3.0 |
| Octyldodecanol | 2.0 | 2.0 |
| C₁₂₋₁₅ Alkylbenzoate | 3.0 | 3.0 |
| Cetearyl Ethylhexanoat + Isopropylmyristate | 2.0 | 2.0 |
| Octocrylene | 2.0 | 2.0 |
| Ethylhexyltriazone | 1.0 | 1.0 |
| Butyl Methoxydibenzoxylmethane | 2.0 | 2.0 |
| Magnesium Aluminium Silicate | 0.2 | 0.2 |
| Glyceryl Glucoside | 2.0 | 3.0 |
| Glycerine | 5.0 | 5.0 |
| Phenoxyethanol | 0.4 | 0.4 |
| Parabene | 0.3 | 0.3 |
| Vitamin E Acetate | 0.1 | 0.1 |
| Hyaluronic Acid | 0.5 | 0.2 |
| NEW INGREDIENT | 0.6 | 0.1 |
| BHT | 0.05 | 0.05 |
| EDTA | 0.2 | 0.2 |
| Carbomer | 2.0 | 0.2 |
| Perfume | q.s. | q.s. |
| Water | ad 100 | ad 100 |

TABLE 8
Retinol comprising shampoo. For further details see text.

| Example: Shampoo | % w/w |
|---|---|
| Sodium Laureth Sulfate | 10.0 |
| Ammonium Lauryl Sulfate | 5.0 |
| Water | 40.0 |
| Cocamidopropyl Betaine | 2.5 |
| Borago Officinalis Seed Oil & | 0.3 |
| Tocopherol & Ascorbyl Palmitate | |
| PEG-40 Hydrogoenated Castor Oil | 1.0 |
| Fragrance | 0.3 |
| Panthenol | 1.0 |
| Disodium EDTA | 0.1 |
| Water | 10.0 |
| Sodium benzoate | 0.5 |
| Citric acid | 0.2 |
| NEW INGREDIENT | 0.05 |
| Sodium Chloride | 2.0 |
| PEG-150 Pentaerythrityl Tetrastearate | 3.0 |
| Water | ad. 100 |

TABLE 9
Retinol comprising anti dandruff shampoo.
For further details see text.

| Example: Anti Dandruff Shampoo | % w/w |
|---|---|
| Aqua (water) | Ad 100 |
| Ammonium laureth sulfate | 10.0 |
| Ammonium lauryl sulfate | 5.0 |
| Glycol distearate | 1.0 |
| Dimethicone | 1.0 |
| Cetyl alcohol | 0.5 |
| Cocamide MEA | 3.0 |
| Zinc Pyrithione | 1.0 |
| Zinc Sulfate | 0.2 |
| TEA-dodecylbenzenesulfonate | 0.2 |
| TEA-Sulfate | 0.15 |
| Guar hydroxypropyltrimonium chloride | 0.2 |
| Hydrogenated polydecene | 1.0 |
| Polyquaternium 10 | 0.3 |
| PEG-7M | 0.5 |
| PEG-40 Hdrogenated Castor Oil | 1.0 |
| Trimethylpropane tricaprylate/tricaprate | 1.0 |
| Pyridoxine HCL | 0.1 |
| Preservative | q.s. |
| Fragrance | 0.3 |
| E 104. E 110. E 132 | 0.02 |
| NEW INGREDIENT | 0.1 |

TABLE 10
Retinol comprising revitalizing hair cream.
For further details see text.

| Example: Revitalizing Hair Cream | % w/w |
|---|---|
| Simmondsia Chinensis (Jojoba) Seed Oil | 3.0 |
| Prunus Armeniaca (Apricot) Kernel Oil | |
| Phenyl Trimethicone | |
| C12-15 Alkyl Benzoate | |
| Glyceryl Stearate SE | |
| Polysilicone-15 | |
| Tocopheryl Acetate | |
| Cetearyl Alcohol | |
| Polyquaternium-7 | |
| NEW INGREDIENT | |
| Aqua | |
| Behentrimonium Chloride | |
| Cocodimonium Hydroxypropyl Hydrolyzed Wheat Protein | |
| Propylene Glycol (and) Diazolidinyl Urea (and) | |
| Methylparaben (and) Propylparaben | |

TABLE 11
Retinol comprising natural lotion. For further details see text.

| Example: Natural Lotion | % w/w |
|---|---|
| ISOPROPYL PALMITATE | 3 |
| PRUNUS AMYGDALUS DULCIS OIL | 1.5 |
| CAPRYLIC/CAPRIC TRIGLYCERIDE | 6 |
| CETEARYL ALCOHOL | 0.6 |
| Helianthus Annuus (Sunflower) Seed Oil | 2 |
| Sodium Benzoate | 0.25 |
| Potassium Sorbate | 0.15 |
| AQUA | Ad 100 |
| CETEARYL GLUCOSIDE | 3 |
| GLYCERIN | 3 |
| Rosa Gallica Flower Extract | 5.0 |
| bentonite | 1 |
| XANTHAN GUM | 0.2 |
| AQUA | 30 |
| LEONTOPODIUM ALPINUM EXTRACT | 2.5 |
| Sodium Hydroxide | 0.3 |
| NEW INGREDIENT | 0.2 |
| TOCOPHEROL | 0.1 |

TABLE 12
Retinol comprising liquid make-up. For further details see text.

| | 1 | 2 | 3 | 4 | 5 | 6 | 7 | 8 |
|---|---|---|---|---|---|---|---|---|
| Example: Liquid make-up | Wt.-% | | | | | | | |
| Water (Distilled) | Ad 100 | | | | | | | |
| Xanthan Gum | 0.3 | 0.33 | 0.33 | 0.1 | 0.33 | 0.33 | 0.33 | 0.33 |
| Cellulose Gum | 0.2 | 0.23 | 0.23 | 0.5 | 0.23 | 0.5 | 0.23 | 0.23 |
| NEW INGREDIENT | 0.3 | 0.1 | 0.1 | 0.15 | 0.25 | 0.01 | 0.1 | 0.05 |
| Triethanolamine | 0.7 | | | | | | | |
| PEG 1450 | 2.35 | | | | | | | |
| Methyl Paraben | 0.28 | | | | | | | |
| Pentylene Glycol | 1.0 | 2.0 | 1.7 | 1.88 | | | | |
| Polysorbate 60 | 0.47 | | | | | | | |
| Titanium Dioxide | 8.44 | 7.0 | 9.0 | 8.5 | 8.44 | | | |
| Brown Iron Oxide | 2.35 | | | | | | | |
| Yellow BC Iron Oxides | 0.94 | | | | | | | |
| Orange Wax | 6.1 | | | | | | | |
| Cera Bellina | 4.69 | | | | | | | |
| Octyl Palmitate | 6.57 | | | | | | | |
| Silicone 245 | 2.0 | 2.0 | | | | | | |
| Cyclopentasiloxane | 1.0 | 1.0 | | | | | | |
| Dimethiconol | 0.20 | | | | | | | |
| Isopropyl Palmitate | 6.1 | 6.1 | 6.1 | | | 1.0 | | |
| C13-15 Alkane | | | 2.0 | 1.55 | 2.8 | | 2.0 | |
| C15-20 Alkane | | | 1.0 | 2.0 | 2.0 | | | |
| C15-19 Alkane | | 6.0 | | | 3.0 | | | |
| Cocunut Alkanes | | | | | | 5.1 | 2.1 | |
| Hydrogenated Castor Oil | 1.41 | | | | | | | |
| Isostearic Acid | 1.5 | | | | | | | |
| Jojoba wax | 0.94 | | | | | | | |
| Dextrin Palmitate | | 4.0 | | | 2.0 | | | 2.0 |

| | |
|---|---|
| Propylene Glycol Stearate | 1.89 |
| Propyl Paraben | 0.28 |

TABLE 13
Retinol comprising moisturizing toner. For further details see text.

| | 1 | 2 | 3 | 4 | 5 | 6 | 7 | 8 |
|---|---|---|---|---|---|---|---|---|
| Example: Moisturizing toner | Wt.-% | | | | | | | |
| Butylene Glycol | 5 | 4 | 3 | 2 | 1 | 2 | | |
| Phenoxyethanol; Ethylhexyl glycerin | | | | | | | 0.8 | 0.5 |
| Caprylyl-Glycol | 0.5 | 0.1 | 0.25 | 0.1 | 0.2 | | | |
| Hexylene Glycol | 1 | 2 | 1.5 | | 1 | 1 | | |
| Propylene glycol | 1 | 2 | 3 | 4 | 5 | | | |
| Dipropylene glycol | | | | | | 1 | 5 | 3 |
| Sorbitol | 1 | 3 | 0.5 | | | | 1 | 1 |
| Panthenol | 0.1 | 1 | 1.5 | | | | | |
| Lactic acid | 1 | 0.5 | | | | | | |
| Sodium Gluconate | 0.05 | 0.1 | 0.2 | | | | | |
| Betaine | 0.5 | 1 | | | | | 0.2 | |
| Sodium Phytate | | | | 0.05 | 0.1 | 0.2 | | |
| Trehalose | | | | 1 | 2 | 0.5 | | |
| Mannitol | | | | | | | 1 | 2 |
| Capryloyl Glycine | | | | | 0.1 | 0.5 | 1.0 | |
| Honey/Mel | | | | 0.1 | 1 | | 2 | |
| alcohol | | | 2 | 1 | | | | |
| Benzyl Alcohol | | | | | | | 0.3 | 1 |
| Hydroxyacetophenone | | | | | 0.5 | 1 | 0.1 | 0.3 |
| Bis-PEG-18 Methyl Ether Dimethyl Silane | 0.1 | 0.2 | 0.5 | 0.15 | | | | |
| Sodium Hyaluronate | 0.01 | 0.2 | | | | | | |
| Biosaccharide Gum 1 | | | 0.1 | 1 | 2 | | | |
| Saccharide Isomerate | 0.5 | | 3 | | 1 | | | |
| Yeast extract | | | 0.01 | 0.2 | | | | |
| Algae extract | 0.01 | | | | | 0.1 | | 0.2 |
| Lactobacillus Ferment Lysate | | 0.3 | | 0.1 | | 0.01 | | |
| NEW INGREDIENT | 0.1 | 0.01 | 0.3 | 0.5 | 0.6 | 0.08 | 0.2 | 0.25 |
| Vitamin E Acetate | 0.1 | | | 0.5 | | 0.2 | 0.5 | |
| Glycerin | 1 | 6 | 2 | 5 | 3 | 4 | 1 | 2.5 |
| Polysorbate-20 | 0.5 | | | | | | | |
| PEG-40 Hydrogenated Castor oil | | | | 0.2 | 0.5 | 0.1 | | |
| PEG-60 Hydrogenated Castor oil | 0.2 | 0.4 | | | | | | |
| PEG-8 | | | | | | | 0.2 | 0.5 |
| Polyglyceryl-6 Caprylate (and) Polyglyceryl-3 | | | | | | | 0.2 | 0.5 |
| Cocoate (and) Polyglyceryl-4 Caprate (and) | | | | | | | | |
| Polyglyceryl-6 Ricinoleate | | | | | | | | |
| Polyglyceryl-3 Caprate/Caprylate/Succinate | | 0.2 | 0.5 | | | | | |

| | |
|---|---|
| Fragrance. Colours | q.s |
| Water | ad 100 |

TABLE 14
Retinol comprising after shave SPF 15.
For further details see text.

| Example: After Shave SPF 15 | % w/w |
|---|---|
| Aqua | Ad 100 |
| Propylene Glycol | 5.0 |
| Butylene Glycol | 5.0 |
| Acrylates/C10-30 Alkyl Acrylate Crosspolymer | 0.3 |
| Disodium Edta | 0.1 |
| Butyl Methoxydibenzoylmethane | 4.0 |
| Octocrylene | 4.0 |
| Polysilicone-15 | 2.0 |
| Ethylhexyl Salicylate | 5.0 |
| Homosalate | 8.0 |
| Neopentyl Glycol Diheptanoate. | 3.0 |
| Propylene Glycol Dibenzoate | |
| Phenoxyethyl Caprylate | 2.0 |
| Dextrin Palmitate, C15-19 Alkane | 3.0 |
| Silica | 2.0 |
| Alcohol Denat.. Aqua | 10.0 |
| Phenoxyethanol. Caprylyl Glycol | 0.5 |
| NEW INGREDIENT | 0.1 |
| Perfume | 0.15 |
| Sodium Hydroxide | qs. |

TABLE 15
Retinol comprising anti-aging serum. For further details see text.

| Example: Anti-aging Serum | % w/w | | | | | |
|---|---|---|---|---|---|---|
| Acrylates/C10-30 Alkyl | 0.3 | | | | | |
| Acrylate Crosspolymer | | | | | | |
| Glyceryl Stearate | 2.5 | | | | | |
| Citrate | | | | | | |
| Coco-Caprylate | 3.0 | | | | | |
| Hydrogenated Coco- | 2.5 | | | | | |
| Glycerides | | | | | | |
| Neopentyl Glycol | 2.0 | | | | | |
| Diheptanoate | | | | | | |
| Dicaprylyl Ether | 2.0 | | | | | |
| Preservative | q.s. | | | | | |
| Glycerin | 2.0 | | | | | |
| Aqua | 82.2 | | | | | |
| Sodium Hydroxide | 0.5 | | | | | |
| NEW INGREDIENT | 0.05 | 0.10 | 0.15 | 0.20 | 0.25 | 0.30 |

TABLE 16
Retinol comprising anti-aging cream. For further details see text.

| Example: Anti- | | | | | | | |
|---|---|---|---|---|---|---|---|
| aging Cream | % w/w | | | | | | |
| Potassium Cetyl | 1.5 | | | | | | |
| Phosphate | | | | | | | |
| Cetyl Alcohol | 2.0 | | | | | | |
| Stearyl Alcohol | 3.0 | | | | | | |
| Hydrogenated Coco- | 4.0 | | | | | | |
| Glycerides | | | | | | | |
| Butyrospermum Parkii | 1.5 | | | | | | |
| (Shea Butter) | | | | | | | |
| Lanolin Alcohol | 1.0 | | | | | | |
| Cetearyl Isononanoate | 1.0 | | | | | | |
| Caprylic/Capric | 2.0 | | | | | | |
| Triglyceride | | | | | | | |
| Ethylhexyl Palmitate | 6.0 | | | | | | |
| C12-15 Alkyl | 5.0 | | | | | | |
| Benzoate | | | | | | | |
| Macadamia Ternifolia | 1.0 | | | | | | |
| Seed Oil | | | | | | | |
| Carbomer | 0.3 | | | | | | |
| Glycerin | 6.0 | | | | | | |
| Butylene Glycol | 3.0 | | | | | | |
| Aqua | ad 100 | | | | | | |
| Palmitoyl Dipeptide-5 | 2.0 | | | | | | |
| Diaminobutyroyl | | | | | | | |
| Hydroxythreonine | | | | | | | |
| & Palmitoyl | | | | | | | |
| Dipeptide-5 | | | | | | | |
| Diaminohydroxy- | | | | | | | |
| butyrate & Glycerin | | | | | | | |
| & Aqua | | | | | | | |
| NEW INGREDIENT | 0.05 | 0.10 | 0.12 | 0.15 | 0.20 | 0.25 | 0.30 |

| | |
|---|---|
| Preservative | q.s. |

TABLE 17
Retinol comprising hand sanitizer gel. For further details see text.

| Example: Hand sanitizer gel | % w/w | | | |
|---|---|---|---|---|
| Aqua | Add 100 | | | |
| Carbomer | 0.8 | 0.7 | 0.4 | 0.7 |
| Ethanol | 70.0 | 60.0 | 65.0 | |
| Isopropanol | | 5.0 | | 70.0 |
| NEW INGREDIENT | 0.05 | 0.10 | 0.20 | 0.03 |
| Tetrahydroxylpropyl | 0.3 | 0.3 | 0.2 | 0.45 |
| Ethylenediamine | | | | |

1. A mixture comprising cis and trans isomers of retinol, wherein the cis/trans ratio within said mixture is 1 to more than 100, wherein the ratio is calculated as wt % of cis-isomers compared to wt % of trans-isomers based on total retinoids in said mixture, particularly wherein the mixture comprises less than 0.3 wt % of cis retinol, based on total retinoids.

2. The mixture according to claim 1, comprising cis- and trans isomers of retinol, with a ratio of 1:105, 1:105.26, 1:110, 1:111.11, 1:117.65, 1:120, 1:125, 1:130, 1:133.33, 1:135, 1:140, 1:142.86, 1:150, 1:153.85, 1:155, 1:160, 1:165, 1:166.67, 1:170, 1:175, 1:180, 1:181.82, 1:185, 1:190, 1:195, 1:200, 1:204.08, 1:205, 1:210, 1:215, 1:220, 1:222.22, 1:225, 1:230, 1:232.56, 1:235, 1:240, 1:245, 1:250, 1:255, 1:260, 1:265, 1:270, 1:275, 1:280, 1:285, 1:285.71, 1:290, 1:300, 1:305, 1:310, 1:320, 1:325, 1:330, 1:333.33, 1:340, 1:345, 1:350, 1:357.14, 1:360, 1:370, 1:380, 1:390, 1:400, 1:420, 1:450, 1:454.55, 1:460, 1:480, 1:500, 1:520, 1:550, 1:555.56, 1:600, 1:666.67, 1:700, 1:800, 1:900, 1:1000, 1:1200, 1:1500, 1:2000, 1:2500, 1:2800, 1:4000, 1:5000, 1:7000 or less, such as 1:1000 or less, more preferably ranges of 1:101 to 1:10000, 1:125 to 1:1000, 1:140 to 1:500, 1:101 to 1:1000, 1:200 to 1:1000, 1:200 to 1:10000, 1:200 to 1:2000, 1:600 to 1:1000, 1:110 to 1:10000, 1:500 to 1:700, 1:150 to 1:400, 1:200 to 1:500, 1:250 to 1:400, 1:250 to 1:1000, most preferably a cis/trans ratio of 1:300 to 1:400.

3. The mixture according to claim 2, wherein the mixture comprising cis- and trans retinol with a ratio of 1:333.

4. The mixture according to claim 1 with a color value of L* being in the range of 50 to 100, a* being in the range of −25 to 10 and b* being in the range of 40 to 150.

5. A pharmaceutical, food, feed, household or cosmetic composition comprising a biologically-derived mixture of claim 1.

6. The composition according to claim 5, wherein the biologically-derived mixture according to any one of claims 1 to 4 is biologically produced through fermentation process.

7. The composition according to claim 5, wherein the mixture has an anthropogenic CO₂ emission profile of zero upon biodegradation.

8. A pharmaceutical or cosmetic composition comprising a mixture according to claim 1, wherein the mixture consists of at least about 95 wt % retinol based on all components in the mixture.

9. The mixture or composition according to claim 1 as an anti-fungal agent.

10. The mixture or composition according to claim 9 as an anti-fungal agent inhibiting the growth of Aspergillus, preferably Aspergillus brasiliensis.

11. The mixture or composition according to claim 1 for the use in a product for improving preservation.

12. The use of a mixture or composition according to claim 1 as anti-fungal or preservative agent in food, cosmetic, pharmaceutical or household products, plastics, paper and/or paints.

13. A method or process of preventing microbial decay and breakdown of food, cosmetic, pharmaceutical and/or household products, plastics, paper and/or paints, wherein said method comprises adding to the products, plastics, paper and/or paints a mixture or composition according to claim 1 as an antimicrobial agent in an amount in the range of 0.005 to 2 wt.-%, based on the total weight of the respective product, plastic, paper and/or paint.

14. A method or process for abolishing and/or inhibiting growth of microbial cells, in particular fungal cells, said method comprising contacting said microbial cells with a mixture or composition according to claim 1, particularly wherein the fungal cells are selected from Aspergillus, preferably Aspergillus brasiliensis.
